(19) United States
(12) Patent Application Publication
(10) Pub. No.: US 2010/0288638 A1
(43) Pub. Date: Nov. 18, 2010

(54) SEPARATION OF BIOMOLECULES

(75) Inventor: Stuart Hassard, London (GB)

Correspondence Address: LANDO & ANASTASI, LLP, ONE MAIN STREET, SUITE 1100, CAMBRIDGE, MA, 02142, US

(73) Assignee: DELTADOT LIMITED, London (GB)

(21) Appl. No.: 12/295,042

(22) PCT Filed: Mar. 29, 2007
(86) PCT No.: PCT/GB2007/050169

§ 371 Date: Aug. 9, 2010

(30) Foreign Application Priority Data
Mar. 29, 2006 (GB) 0606181.6

(51) Int. Cl. C07K 1/28; G01N 27/26

(52) U.S. Cl. 204/461; 204/456; 204/644; 204/610; 204/612; 204/606

(57) ABSTRACT

A method and apparatus for performing separation of molecules, in particular biomolecules such as nucleic acids and peptides, are disclosed. The method comprises separating molecules by a first characteristic along a linear dimension of a channel, and separating the molecules by a second characteristic along the same linear dimension, with the rust and second separations being carried out within at least partially overlapping sections of the channel. The two separations may then be combined to give a virtual two-dimensional separation which is carried out in a single dimensional real space. The method may also include detecting molecules during the second separation, preferably using an intrinsic characteristic of the molecules for example UV absorbance.

### FIELD OF THE INVENTION

The present invention relates to methods and devices for the separation of molecules, in particular biomolecules, and more particularly proteins and peptides. The invention is directed to the separation of molecules on the basis of two or more characteristics of the molecules; in preferred embodiments these characteristics are isoelectric point and charge/mass ratio.

### BACKGROUND OF THE INVENTION

Protein separation and analysis is a key method in molecular biology, given added significance by the rise of proteomics, whereby the intention is to study the total protein content of whole cells. Two dimensional (2D) analysis of proteins allows the separation of proteins according to two characteristics of the molecules, typically electric charge and molecular weight. The proteins will be separated firstly by isoelectric focusing along a first dimension of a two dimensional gel, and secondly by polyacrylamide gel electrophoresis (PAGE) along a second dimension, orthogonal to the first. This dual separation allows isolation of different proteins that share a common property; for example, two proteins of the same molecular weight but with a different charge.

2D maps of proteins from whole cells have been developed for many applications in proteomics such as monitoring of the intensity of a particular protein as a marker of disease progression. It is important to note the migration of any given protein through a gel is affected by its environment. The presence of attached marker molecules can alter its movement profile.

The first separation is by isoelectric focusing, which uses the intrinsic charge properties of proteins to position them on a gel. In a typical protocol, the tissue or cell culture sample is prepared and run on a 5% acrylamide pH gradient gel for 21 hrs (˜8.8 Kv) until each protein has migrated to the position on the gel where its net charge is neutral. The proteins are then separated by sodium dodecyl sulphate polyacrylamide gel electrophoresis (SDS-PAGE) for ˜7 hrs. Unlabelled gels are visualised by staining, for example with Coomassie Blue. The addition of stains to visualise the protein positions at this point increases the complexity of further manipulations on identified molecules. In order to investigate changes in protein expression in response to external stimuli, such as stimulation of a cell with cytokines or other factors, two dimensional electrophoresis may be carried out on samples before and after stimulation, and the two gels compared. Upregulation of protein expression should be apparent as a change in intensity in a particular protein spot.

2D electrophoresis is a very powerful and established technique for the separation of macromolecules. However, there are a number of drawbacks to conventional separation techniques. When comparing two different gels, the amount of perceived alteration in any protein is dependent on the accuracy of the technique. This data is obtained by staining the gel matrix with a dye such as Coomassie Blue. Staining gels to visualise the proteins in them is an error prone process as the process itself affects the apparent intensity of the proteins. Therefore small errors in the staining process can lead to large errors in the gel where the induction or suppression of the protein can be an artefact.

The gels are difficult to set up, run and are inherently analogue; high levels of operator skill and knowledge are required to use the systems.

Data analysis is complicated by the need for complex image collection, digitisation and peak location which results in insufficient resolution and unreliable quantification.

The entire process is labour intensive and is difficult to automate, which produces variances in results due to operator and equipment differences. Typical 2D runs take 24 hours, and where automated systems are available, they may be considered unreliable.

The present invention provides an alternative method and devices for separation of molecules. Certain embodiments of the invention make use of label-free intrinsic imaging (LFII). Rather than incorporating external dyes and markers into a molecular separation, LFII relies on inherent characteristics of the molecules being separated for detection. A preferred characteristic is absorbance of UV light. We have previously described use of LFII in international patent applications WO03/036302 and WO03/102238, which disclose detection of nucleic acids and proteins. We have also previously described use of LFII in combination with molecular separation techniques to fractionate molecules by charge/mass ratio within a microfluidics channel. These techniques are described in, for example, WO96/35946 and WO02/12876. In these publications, the technique is described of separating molecules by electrophoresis, and detecting the molecules as they travel through a microfluidics channel past a series of detectors. The detected data are used to determine the velocities of the molecules, and hence to extrapolate the molecular weight or other characteristic of the molecules. The disclosures of all publications mentioned herein are incorporated by reference.

The present invention makes use, in certain embodiments, and in part, of the separation and detection techniques referred to above.

### SUMMARY OF THE INVENTION

According to a first aspect of the present invention, there is provided a method of separating molecules in accordance with at least two characteristics of the molecules, the method comprising the steps of:
separating molecules according to a first characteristic within a first section of a channel in a first linear dimension;
separating said molecules according to a second characteristic within a second section of the channel in said first linear dimension;
wherein said first and said second separations are carried out within at least partially overlapping sections of the channel.

The present invention thus provides a “virtual 2D separation”, as the molecules are separated by two characteristics across a single linear dimension. The first separation allows the second separation to be carried out on molecules at different starting origins, such that the outcome of the second separation depends in part on the first separation. The first characteristic is preferably charge, and the method comprises the step of isoelectrically focusing the molecules within the first section. For example, an electric field may be applied to molecules within a pH gradient established within the first section.

The second characteristic is preferably charge/mass ratio, and the method comprises the step of separating the molecules by application of an electric field across the second section.

The method preferably further comprises the step of detecting molecules during the second separation. Preferably the molecules are detected as they are separated; for example, as the molecules pass a particular point within the channel. The detection may be carried out using labels incorporated into the molecules, but preferably the molecules are detected using an intrinsic characteristic of the molecules. Conveniently the method comprises the step of illuminating a detector, preferably a UV detector, with a light source, preferably a UV light source, and detecting molecules as the pass between the detector and the source by the absorbance of the light by the molecules.

Preferably the molecules are detected as they pass a plurality of separated points; the points may be located along substantially all of the length of the second section, and preferably along substantially all of the length of the channel. The molecules may be detected during the first separation as well as during the second separation.

The method may further comprise the step of determining the velocities of detected molecules during separation. The velocities may be determined by extrapolating the detected locations of molecules to give an equiphase space-time map. The method preferably also comprises the step of calculating the first and second characteristic, preferably the charge/mass ratio and isoelectric point, for the molecules based on the determined velocities. This may be done by, for example, using the determined velocities of molecules to extrapolate the position of each molecule at time zero; that is, the time at which the second separation began. At time zero, the location of each molecule is its isoelectric point, assuming that the first and second characteristics are isoelectric point and charge/mass ratio respectively. The velocity itself is characteristic of the charge/mass ratio of the molecule.

The method may yet further comprise the step of generating a graph of the first characteristic against the second characteristic for each detected molecule. This provides a “virtual” 2D electrophoretic map of the separated molecules.

The method may comprise the step of separating a plurality of mixtures of molecules in a plurality of channels.

According to a further aspect of the invention, there is provided an apparatus for use in separating molecules, the apparatus comprising:
a channel having first and second sections, said sections at least partially overlapping;
the first section being adapted to allow separation of molecules within the section by a first characteristic along a first linear dimension; and
the second section being adapted to allow separation of molecules within the section by a second characteristic along said linear dimension.

The apparatus allows molecules to be separated by two characteristics along the same linear dimension. The separations may be carried out in opposite directions; for example, the molecules may move back and forth within the channel. The channel may be curved, serpentine, coiled, looped, or the like. That is, although the separation is carried out in a linear dimension, the channel need not be strictly straight. Introducing bends or curves into the channel allows the effective length of the channel to be increased without greatly increasing the volume necessary to contain the channel.

The molecules are preferably biomolecules, more preferably biological polymers, and most preferably peptides or proteins. The two terms ‘peptide’ and ‘protein’ are used interchangeably herein, except where otherwise indicated.

The first section preferably overlaps at least half its length with the second, more preferably at least three quarters, and most preferably the first section is substantially completely or completely contained within the second section.

The first characteristic is preferably charge, and the first section is adapted to allow isoelectric focusing of the molecules within the section. The first section preferably contains a pH gradient. The gradient may be established for example by one or more semi-permeably membranes dividing fluid regions of differing pH from one another; or may be established by a pH gradient established within a solid matrix such as a gel. Alternatively, the first section may be located adjacent a series of chambers containing buffer at a range of different pH; the chambers may be separated from the first section by a semi-permeable membrane, such that a pH gradient is established within the section. The first section preferably comprises electrodes for carrying out a separation, and is preferably delimited by said electrodes.

The second characteristic is preferably charge/mass ratio. The second section preferably comprises electrodes, and may be delimited by said electrodes. At least one of the electrodes of the second section may be shared with the first section, such that at least one electrode is common to the first and second sections.

The apparatus preferably further comprises means for detecting separated molecules. The molecules may be labelled to allow detection, although preferably the molecules are unlabelled, and an intrinsic characteristic of the molecules is detected. In preferred embodiments, the absorbance of light, preferably UV light, by the molecules is detected. The detector means in such embodiments may comprise a UV light source and a UV detector, arranged such that the channel is interposed between the source and the detector. A plurality of detectors may be provided; preferably the detectors are spaced along the length of the channel, and more preferably spaced along substantially the whole length of the channel.

The apparatus preferably also comprises means for determining velocities of molecules being separated. For example, the apparatus may comprise an equiphase space-time map generator for generating an equiphase space-time map of equiphase points from data sets representative of detected molecules at a plurality of spaced positions along the channel. Such a system is described in WO02/12876, the contents of which are incorporated herein by reference. Determination of velocities of separating molecules allows the calculation of the charge/mass ratio and isoelectric point for each molecule, using this or a similar system. An alternative system for determining velocities of migrating molecules is described in WO96/35946. The apparatus may comprise means for extrapolating the position of the molecules at the start of the second separation from the determined velocities.

The apparatus may still further comprise means for generating a graph of the first characteristic against the second characteristic for each detected molecule.

The channel is preferably adapted to denature proteins therein; for example, the channel may comprise a denaturing gel, such as SDS-polyacrylamide. The channel may also or instead be adapted to affect interactions between molecules; for example, the channel may be temperature controlled; a high temperature may be used to denature proteins or nucleic acids, while a low temperature may be used to reduce protein activity.

The apparatus may comprise a plurality of channels. This allows separation of multiple molecules simultaneously; this may be used for high throughput screening, or may be used for direct comparison of two samples, for example, protein content of a cell sample before and after stimulation.

### BRIEF DESCRIPTION OF THE DRAWINGS

FIG. 1 shows a schematic illustration of a device for separating molecules in accordance with an embodiment of the present invention.

FIG. 2 shows a representation of determining the charge/mass ratio and isoelectric point for detected molecules; and

FIG. 3 shows a simulation of a plot of charge/mass ratio against isoelectric point for a plurality of proteins, representative of the results which may be obtainable using the method of the present invention.

### DETAILED DESCRIPTION OF THE DRAWINGS

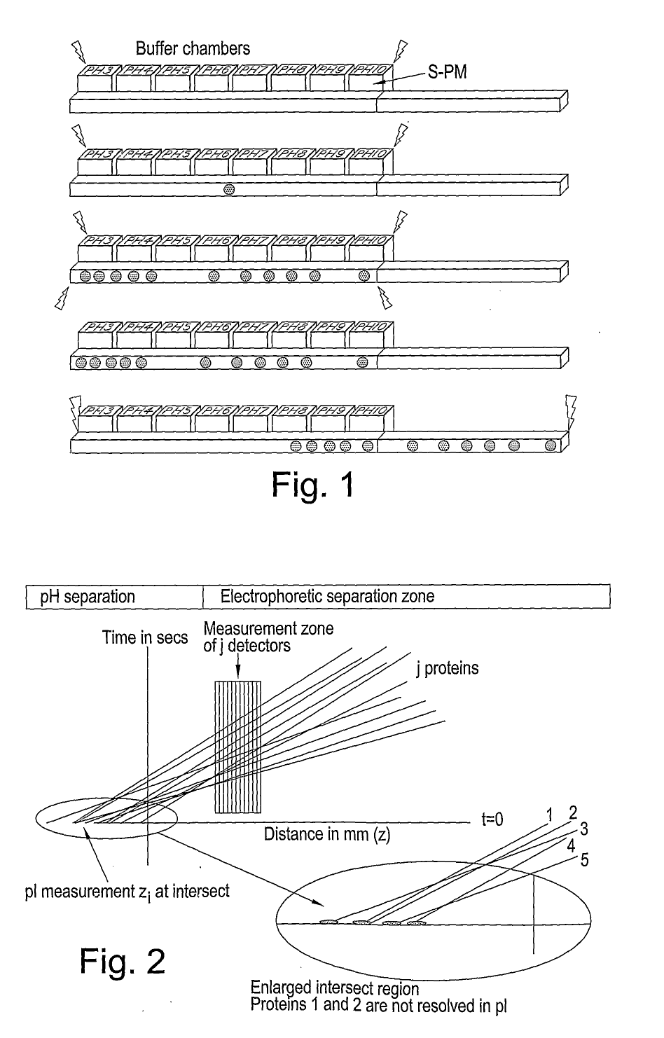

FIG. 1 shows a schematic illustration of an apparatus for separating molecules in accordance with the present invention. The principle of the invention involves the use of a chip in which an electrophoretic separation channel has been fabricated. At the start of the channel there is an isoelectric focusing section containing a pH gradient gel. The pH gradient could be induced across a semi-permeable membrane and controlled by electric fields projected by embedded electrodes. Molecules to be separated are placed in the focussing section and move to an equilibrium position under the influence of another electric field. The positioning and control of these electrodes will be of paramount importance as the electric fields generated will be complex. After the focussing, which will be achieved in minutes rather than hours, the electrophoretic state of the molecules is then changed and a high voltage potential applied across the entire length of the focussing and separation sections. The molecules migrate under the influence of the electric field from the focussing section into the separation section where their position and velocity are tracked. Tracking is typically carried out using label free intrinsic imaging (LFII), in which UV absorbance of molecules is determined as they pass a UV light source and detector; a change in intensity of detected light indicates that a band of molecules is passing the detector.

Using this information it will be possible to determine the charge/mass ratio and the isoelectric point for each molecule. This can be done using advanced signal processing algorithms, such as those described in our earlier patent applications WO02/12876 and WO96/35946. A representation of the determination of this information is shown in FIG. 2. Proteins are separated according to their charge/mass ratio, and their velocity determined as they pass detectors located along the channel. Using the known position (from the position of the detectors) and the known velocity, their position at time t=0 is calculated. This position at t=0 represents their isoelectric point, which the proteins have reached after the first separation. In the example shown in FIG. 2, note that proteins 1 and 2 are not resolved by their isoelectric point (pI), but that does not mean the value obtained for pI is incorrect: they happen to be degenerate. Note also that it is very unlikely to have both pI and Mw (molecular weight, or charge/mass ratio) coincident.

Furthermore, if the chip carries multiple identical channels it should be possible to make direct and rapid comparisons between samples.

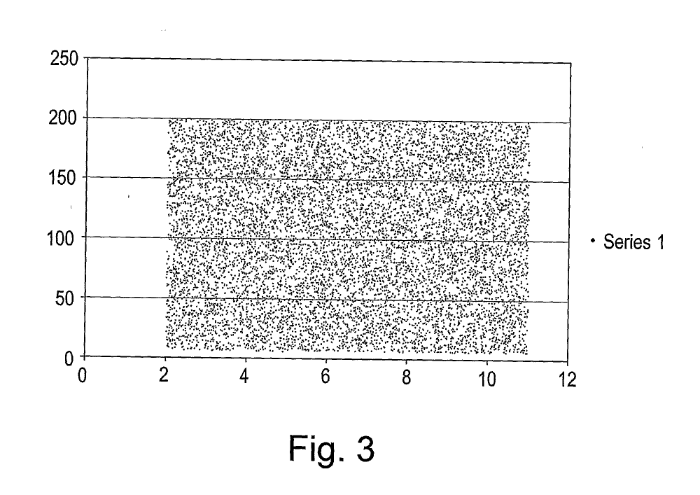

This method will allow 2-dimensional levels of data to be acquired from a 1-dimensional microfluidic system—This is Virtual 2-Dimensional (V2D) electrophoresis. A two dimensional map can be plotted of charge/mass ratio against isoelectric point for a range of proteins from a sample. FIG. 3 is a simulation of such a map, showing 10,000 protein dots in. V2D output showing Mw in kDa plotted against pI. This level of detail will be very difficult to achieve, a typical number of proteins which can be resolved in practical terms may range from 10 (typical ladder) to 100s (typical cell lysate). The resolutions, sensitivities and quantification of the methods described herein far exceed those possible in gels.

In certain situations, the protein bands can influence each other—this is more likely to be the case in the protein bands which have great clinical significance—for example those with post-translational modifications. Bands passing through each other may interfere and cause errors in the determination of Mw (from the slope), and errors in the back-extrapolation to get pI. The solution may be a combination of good chemistries—SDS will denature most of the proteins and allow them to pass more easily through one another; high electric fields: residual protein-protein interactions may be made negligible by using very high electric fields; use of temperature controls to minimize the inherent protein interaction; and interesting geometries: band-band interactions may be minimized by keeping the separation lengths long—particularly the first separation. Any or all of these strategies may be used to reduce protein interactions.

Further, monitoring the proteins throughout large parts of their separation may be used. By imaging their entire journey, we may be able to monitor and correct for any band-band interactions.

### The Benefits of the System.

While fundamental advances in proteomics mean that the ability to characterise proteins and link this analysis to disease states is improving exponentially there are several bottlenecks which will have to be addressed before this knowledge can be translated into viable patient care options. While we will soon be able to accurately target more defined groups of patients who will react effectively to a specific range of tailored therapeutic options, the overall cost benefit of carrying out such analysis diminishes as each therapeutic option is restricted to a smaller patient base, diminishing returns on R&D and increasing management costs of therapeutic care. One key bottleneck in the system is that of initial analysis of complex protein samples taken from patients. Present methodologies are expensive, time consuming and technically specialised. As a result this type of analysis is mainly confined to academic, large industrial, and a few specialised hospital settings. Our technology will address all of these issues through the use of technology which dramatically reduces time to result, reduces the technical specialism and uses simple but very powerful algorithms to reduce instrumentation costs, thus lowering the barrier to adoption within the wider healthcare community.

Label Free Intrinsic Imaging (LFII) is a novel imaging system that uses no chemical labels to detect biomolecules during their separation by electrophoresis. LFII requires advanced signal processing and pattern recognition tools to compensate for the huge loss in signal that having no label entails. This is achieved by using algorithms directly adapted from high-energy particle physics, and described in our earlier patent applications. Our instruments may use capillaries as the separation system, but we prefer to perform both nucleic acid and protein separations on chips, which have advantages in speed of separation, increased resolution and physical size. It is the combination of no labels, advanced signal processing and microfluidics that gives LFII its importance. Benefits include:

Health and safety benefits—no intercalating chemicals or radioactivity. No disposal considerations.
Relative quantification—+1% accuracy on measurement of relative concentrations of molecules. Our proprietary signal processing uses peak height—rather than area—information for quantification.
Resolution—better than 400 Da at 15 kDa molecular weight. We get excellent reproducibility of results.
Sensitivity—1 μg/ml detectable over 6 kDa to 200 kDa molecular weight range.
Speed of analysis: Rapid through-put of samples with rapid sample injection times.

Easy sample loading and ruggedisation capability means great ease of use and reliability.

Results are generated in intrinsically digital format facilitating automated analysis. Powerful suite of data handling and analysis tools allowing “All In One” results analysis. Great ease-of-use for routine user and profound analytical insights for specialist or power user.

Within electrophoretic techniques by far the most common method is 2D gel electrophoresis; despite this popularity the technique has changed little in 25 years of use and remains a technique that is complex manual process to execute (over 9 steps are required) time consuming, difficult to make reproducible (both due to reagents and instrument), prone to errors and most importantly unpopular method with all those that perform it

There is a need for a technology that simplifies, automates and reduces the costs associated with 2D electrophoresis but delivers the same approximate performance and cost per separation/analysis. The microfluidic electrophoretic device and methods described herein can help to meet this demand. We aim to provide on chip isoelectric (TEF) focusing (pH gradient); charge mass separation on the same channel as the 1EF; an integrated polymer substrate that contains the above; real time system analysis and control software algorithms. The resultant device will enable fully automatic, high resolution protein separation within a disposable chip. The chip format will then enable a desktop instrument to drive the system, handle fluids and provide the results.

1. A method of separating molecules in accordance with at least two characteristics of the molecules, the method comprising the steps of:
separating molecules according to a first characteristic within a first section of a channel in a first linear dimension;
separating said molecules according to a second characteristic within a second section of the channel in said first linear dimension;
wherein said first and said second separations are carried out within at least partially overlapping sections of the channel.

2. The method of claim 1, wherein the first characteristic is charge.

3. The method of claim 1 or claim 2 wherein the method comprises the step of isoelectrically focusing the molecules within the first section.

4. The method of any preceding claim wherein the second characteristic is charge/mass ratio.

5. The method of any preceding claim wherein the method comprises the step of separating the molecules by application of an electric field across the second section.

6. The method of any preceding claim further comprising the step of detecting molecules during the second separation.

7. The method of claim 6 wherein the molecules are detected as they are separated.

8. The method of claim 6 or 7 wherein the molecules are detected as they pass a particular point within the channel.

9. The method of any of claims 6 to 8 wherein the molecules are detected using an intrinsic characteristic of the molecules.

10. The method of claim 9 comprising the step of illuminating a detector, preferably a UV detector, with a light source, preferably a UV light source, and detecting molecules as they pass between the detector and the source by the absorbance of the light by the molecules.

11. The method of any of claims 6 to 10 wherein the molecules are detected as they pass a plurality of separated points.

12. The method of claim 11 wherein the separated points are located along substantially all of the length of the second section.

13. The method of claim 11 wherein the separated points are located along substantially all of the length of the channel.

14. The method of any preceding claim comprising the step of detecting the molecules during the first separation.

15. The method of any of claims 6 to 14 comprising the step of determining the velocities of detected molecules during separation.

16. The method of claim 15 wherein the velocities are determined by extrapolating the detected locations of molecules to give an equiphase space-time map.

17. The method of any of claims 6 to 16 comprising the step of calculating the first and second characteristic of the molecules.

18. The method of any of claims 6 to 16 comprising the step of calculating the charge/mass ratio and isoelectric point of the molecules based on the determined velocities.

19. The method of claim 18 wherein the calculating is done by using the determined velocities of molecules to extrapolate the position of each molecule at the time at which the second separation began.

20. The method of any of claims 17 to 19 comprising the step of generating a graph of the first characteristic against the second characteristic for each detected molecule.

21. The method of any preceding claim further comprising the step of separating a plurality of mixtures of molecules in a plurality of channels.

22. The method of any preceding claim wherein the molecules are peptides or proteins.

23. An apparatus for use in separating molecules, the apparatus comprising:
a channel having first and second sections, said sections at least partially overlapping;
the first section being adapted to allow separation of molecules within the section by a first characteristic along a first linear dimension; and
the second section being adapted to allow separation of molecules within the section by a second characteristic along said linear dimension.

24. The apparatus of claim 23 wherein the channel is curved, serpentine, coiled, looped, or the like.

25. The apparatus of claim 23 or 24 wherein the molecules are peptides or proteins.

26. The apparatus of claims 23 to 25 wherein the first section is substantially completely or completely contained within the second section.

27. The apparatus of claims 23 to 26 wherein the first section is adapted to allow isoelectric focusing of the molecules within the section.

28. The apparatus of claim 27 wherein the first section contains a pH gradient.

29. The apparatus of claim 28 wherein the pH gradient is established within a solid matrix such as a gel.

30. The apparatus of claims 23 to 29 wherein the first section comprises electrodes for carrying out a separation.

31. The apparatus of claims 23 to 30 wherein the second section comprises electrodes.

32. The apparatus of claim 31 when dependent on claim 30, wherein at least one of the electrodes of the second section is shared with the first section.

33. The apparatus of claims 23 to 32 further comprising means for detecting separated molecules.

34. The apparatus of claim 33 wherein an intrinsic characteristic of the molecules is detected.

35. The apparatus of claim 33 or 34 wherein the detector means comprises a UV light source and a UV detector, arranged such that the channel is interposed between the source and the detector.

36. The apparatus of claims 33 to 35 wherein a plurality of detectors is provided.

37. The apparatus of claim 36 wherein the detectors are spaced along the length of the channel.

38. The apparatus of claims 23 to 37 comprising means for determining velocities of molecules being separated.

39. The apparatus of claim 38 comprising an equiphase space-time map generator for generating an equiphase space-time map of equiphase points from data sets representative of detected molecules at a plurality of spaced positions along the channel.

40. The apparatus of claim 38 or 39 comprising means for extrapolating the position of the molecules at the start of the second separation from the determined velocities.

41. The apparatus of claims 23 to 40 comprising means for generating a graph of the first characteristic against the second characteristic for each detected molecule.

42. The apparatus of claims 23 to 41 wherein the channel is adapted to denature proteins therein.

43. The apparatus of claims 23 to 42 wherein the channel is adapted to affect interactions between molecules.

44. The apparatus of claims 23 to 43 wherein the channel is temperature controlled.

45. The apparatus of claims 23 to 44 comprising a plurality of channels.
